(19) United States
(12) Patent Application Publication
(10) Pub. No.: US 2004/0122298 A1
(43) Pub. Date: Jun. 24, 2004

(54) Method and apparatus for deriving information from physiological responses

(76) Inventor: Alexander I. Poltorak, Monsey, NY (US)

Correspondence Address: General Patent Corporation, Montebello Park, 75 Montebello Road, Suffern, NY, 10901, US

(21) Appl. No.: 10/328,822

(22) Filed: Dec. 24, 2002

(51) Int. Cl. A61B 5/00

(52) U.S. Cl. 600/300

(57) ABSTRACT

Decision-making may be facilitated and subconscious information may be elicited from a subject by questioning the subject while monitoring the subject's physiological responses to the questions.

### FIELD OF THE INVENTION

The present invention pertains to information management, decision making, and, more particularly, to a method of utilizing an individual's physiological responses to questions to elicit subconsciously held information and to harness an individual's intuitive knowledge and problem-solving skills to the solution of complex problems.

### BACKGROUND

Research indicates that in a complex and fluid environment, an intuitive approach to decision making is preferable. Brokers working on stock exchanges and day-traders are known to make split-second decisions based on their intuition rather than an elaborate analysis. Doctors working in hospital emergency rooms often have to make instant life and death decisions based on their “gut feeling”. Military commanders, who often find themselves on a battlefield in the midst of a rapidly changing, complex situation, are known to follow their instinct honed by the years of training and combat experience. The US Marines Corps recently adapted this intuitive decision making as their official training doctrine. Good managers are also known to follow their instincts. However, many decision makers, in management and other fields, find it difficult to tap into their subconscious mind, to get in touch with their instinct or intuition.

Research indicates that individuals exhibit various subtle detectable physiological responses indicative of their intuitive reactions to various situations, questions and other stimuli. Even where they have no conscious knowledge on which to base an objective response, physiological responses are triggered by the subconscious mind. Such responses are popularly known as “gut reaction”, “gut instinct”, perception, insight, instinct, “sixth sense”, or simply “intuition.” “Intuition” is, in fact, the direct, nonverbal solution of problems by the subconscious mind.

Often, decision-makers are faced with the need to solve extremely complex problems, or to solve problems on the basis of imperfect, incomplete or uncertain information. The optimal solution of these problems is difficult to achieve by traditional methods. Various decision-making tools, such as linear optimization models, decision tree analysis, Monte Carlo simulation and other tools are complex and time consuming. More importantly, as approximation models, they inevitably miss all of the intangible, not easily quantifiable factors, let alone the decision-maker's instinct. On other occasions, individuals may be asked to disclose information of which they are not consciously aware, but which they do possess, albeit at a subconscious level.

Everybody has intuition, based in part on prior experience, although, most people are not cognizant of it. An intuitive reaction is typically associated with subtle but detectable physiological responses of the body. Such physiological responses may include:

Increase in the rate, rhythm, and strength of the heartbeat;

Elevated blood pressure;

Rerouting of blood flows in the circulatory system;

Change in electrical conductance of the skin;

Change in the shape of the pulse wave;

Rise in the blood sugar level;

Increased metabolic rate;

Dilation of the bronchi and pupils;

Hair standing on end (“gooseflesh”);

Reduction of blood clotting time; and

Increased ACTH secretion by the pituitary gland.

Several of these responses may be conveniently detected by existing types of monitoring equipment. Indeed, conventional “lie detector” machines are examples of such monitoring equipment which are intended to detect the physiological response to the disparity, if any, between a subject's verbal response to a question and his knowledge as to the correct or “true” answer. It is important to note, however, that the physiological response to intuitive or subconscious knowledge is less pronounced than the corresponding reaction to a lie in a lie-detector test.

The present invention is based on the notion of psychosomatic memory, which stores in the subconscious mind the memory of somatic and psychological responses to certain internal and external stimuli and information. Said Psychosomatic subconscious memory is often associated with intuition, instinct, perception, insight, “sixth sense”, “body memory”, or “gut feeling”.

Among the different autonomic responses, the most useful and the best studied is the skin conductance response. Skin resistance is recognized as distinctively sensitive to transitory emotional states and mental events. It is, therefore, a preferred medium for the detection and measurement of physiological response to a question. However, because such responses tend to be muted, a highly sensitive system, employing active electrodes, may be required.

### SUMMARY OF THE INVENTION

Heretofore, no conscious, planned, methodical or organized use has been made of these subconsciously triggered physiological responses other than for the aforementioned purpose of detecting lies.

It is, therefore, a primary object of the preset invention to provide a method of eliciting subconscious knowledge from an individual.

It is a further object to provide a method of harnessing subconscious knowledge for the solution of what may otherwise be intractable problems.

The above and other objects and advantages of the present invention as may hereinafter appear are achieved by questioning a subject while monitoring the subject's physiological responses to the questions so as to determine the subject's subconscious or intuitive knowledge. The information elicited in such manner may be utilized in the solution of complex problems wherein complete or accurate information is lacking.

### BRIEF DESCRIPTION OF THE DRAWINGS

FIG. 1 is a flow chart of a method in accord with the present invention;

FIG. 2 is a flow chart of another method in accord with the present invention;

FIG. 3 is a flow chart of yet another method in accord with the present invention.

FIG. 4 is a pictorial representation of an apparatus in accord with the present invention.

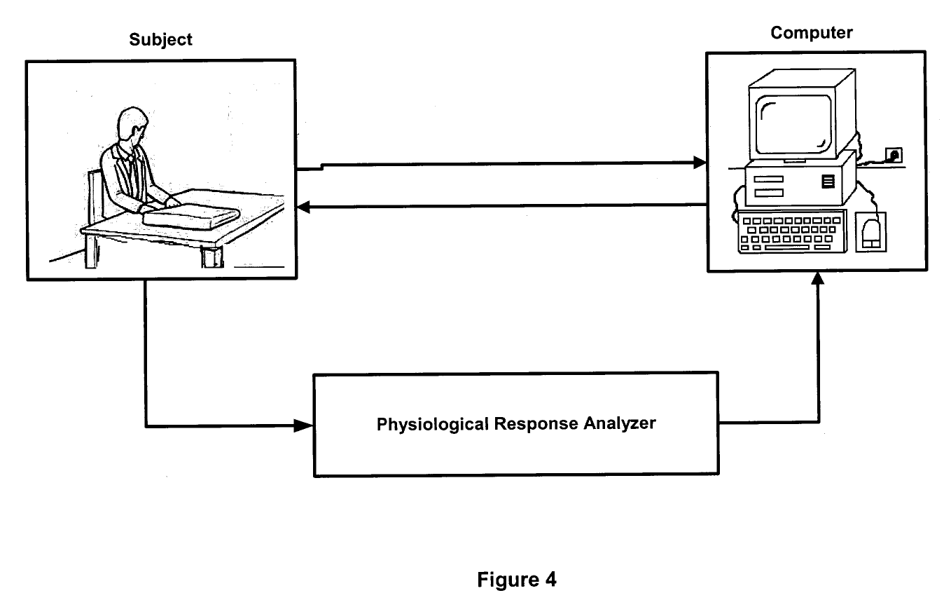

### DESCRIPTION OF THE PREFERRED EMBODIMENTS

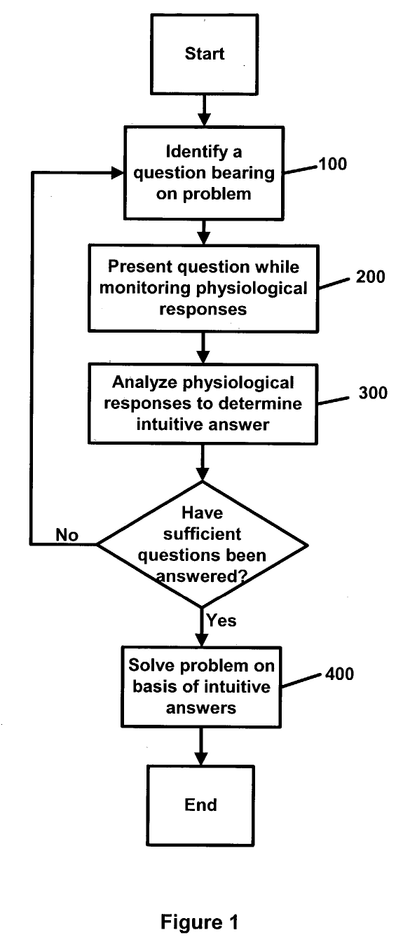

As visually presented in FIG. 1, a problem may be solved in accord with the present invention, by identifying one or more questions pertaining to the subject problem, the answers to which would bear on the solution to the given problem. In the flow chart, this is seen as step 100. Thus, for example, if the problem to be solved was whether to invest in a given start-up company, the questions pertaining thereto might be directed to, among other things, the level of expertise of the company's management team, cost and sales projections, competition, current financial condition of the company, and general economic outlook. This example is more fully developed below. While the problem to be solved involves the interplay of many factors, the individual questions are selected so as to be as single-faceted as possible. Moreover, the questions should be structured to elicit clear, definitive answers, i.e. YES/NO, HIGH/LOW, rather than narrative or vague answers.

The questions are presented at step 200 to the problem-solver who is being monitored so as to detect and, if desired, to record physiological responses to the questions. As hereinabove set forth, the subconscious mind will trigger a variety of physiological responses to these questions. Many of these responses, such as pulse rate and blood pressure, can be detected and measured with presently available equipment. Others, such as dilation of the pupils and hair standing on end, can often be detected by simple visual observation of the subject. By monitoring the problem-solver's physiological responses to the questions, his subconscious responses thereto can be determined.

As would be apparent to those who watch crime programs on television, it may be necessary or advantageous to begin with a few innocuous questions to establish a baseline before presenting substantive questions. Once a baseline has been established, the substantive questions are sequentially presented and the problem-solver's physiological response noted.

The questions to be asked during an interview session should be grouped into several lines of questioning. If a subject exhibits no noticeable reaction, it may be advisable to switch to another line. Conversely, if the subject is exhibiting a noticeable reaction to a given line of questioning, such line should be pursued to a greater depth. It is advisable to pursue such line of questioning for a while, then switch to another line, only to come back to the line of questioning to which the subject has exhibited a noticeable reaction before.

The following is an example of an interview with a venture capitalist facing a decision to invest or not to invest in a start up company. The venture capitalist (“VC”) has completed his due diligence on the products, markets and the management team of the new venture he is asked to invest in. Given the numerous risk factors present and the high degree of uncertainty associated with estimating any of these factors, this decision does not lend itself easily to an analytical approach. The first task is to formulate the problem.

The question is whether to invest in a start-up venture which is associated with the following risk factors:

1. R&D Risk: whether the company will be able to develop a working prototype of their product.

2. Patentability Risk: whether the novel element of the company's product will be patentable.

3. Production Risk: whether the company will be able to mass produce (or find a contract manufacturer) the new product at an acceptable price level.

4. Market Risk: whether the marketplace will accept the new product with enthusiasm, i.e. whether the new product will be successful in the marketplace.

5. Management Risk: whether the founders of the new venture (the techies), will be able to mature into good managers capable of growing the company into a new phase.

6. Financial Risk: whether the new venture will be able to find further sources of funding for subsequent rounds of venture financing.

7. Exit Risk: whether the VC will have an exit vehicle by way of an IPO or sale of the company.

Each if these risk factors requires its own line of questioning. The following is an example of the questions pertaining to this investment decision problem:

Base-line questions:

Is your name Joe Smith?

Are you wearing a tie today?

Is today Tuesday?

Did you watch a movie last weekend?

Do you have children?

Do you like ice-cream?

Do you like chocolate?

Do you like looking at the ocean?

1. R&D Risk:

(a) Do you like the idea behind the product?

(b) Did you like the presentation?

(c) Was it exciting?

(d) Is the technology novel?

(e) Does the Venture have a working prototype?

(f) Do you think they will be able to make it work?

(g) Will they do it on time?

(h) Are these guys good at what they do?

(i) Do they know their technology?

(j) Do you consider them experts in this field?

(k) Are confident they will make it work?

2. Patentability Risk

(a) Does the technology appear to you as novel?

(b) Does it appear to you obvious?

(c) Did you have a patent attorney look at it?

(d) Did the patent attorney say it was patentable?

(e) Was the patent attorney certain it was patentable?

(f) Does the Venture have any patents already?

(g) Do they plan to file for a patent?

(h) Do they have the money to file for a patent?

(i) Are you willing to give them money to file for a patent?

(j) Do you want to file for a patent?

3. Production Risk

(a) Does this product require manufacturing?

(b) Can this product be manufactured in the existing facility?

(c) Does it require building a factory?

(d) Will the Venture have enough money to build the factory?

(e) Does the management have any experience in manufacturing?

(f) Can the manufacturing be contracted out?

4. Market Risk:

(a) Do you like the product?

(b) Would you buy it?

(c) Would you wife buy it?

(d) Do you think the world needs it?

(e) Is there another product on the market that solves the same need?

(f) Is it successful?

(g) Do you think this product will successfully compete in the marketplace?

(h) Do you think people will fall in love with this product?

(i) Is it a consumer product?

(j) Will it be expensive to market?

(k) Will the Venture have enough money to fund marketing campaign?

(l) Do the founders have any experience in marketing?

(m) Are you confident the product will be successful in the marketplace?

5. Management Risk

(a) Have you met the founders yet?

(b) Did they impress you?

(c) Did you like them?

(d) Are they smart?

(e) Are they arrogant?

(f) Are they technology-savvy?

(g) Are they market-savvy?

(h) Are they immature?

(i) Do they communicate well their vision?

(j) Do they make you comfortable?

(k) Are they experienced in marketing?

(l) Are they experience in sales?

(m) Are they experience in production?

(n) Are they experienced in management?

(o) Do you expect them to learn all this?

(p) Do you think they will mature into good businessmen?

(q) Have they been upfront with you?

(r) Are they honest?

(s) Are they going to run off with your money?

(t) Do you like them?

(u) Do you have confidence in them?

6. Financial Risk

(a) Does the Venture have any money now?

(b) Are they going to close down if you do not invest?

(c) Do you want to invest?

(d) Do you know that it will take probably twice as much as you expect to get them to the next stage?

(e) Have you set aside twice as much as you are planning to invest?

(f) Do you know they will likely to run out of money much faster than they expect?

(g) Are you prepared to invest more to keep them going?

(h) Will you invest more if they meet the milestones?

(i) Do you have enough money to invest more?

(j) Do you have other sources of funds for the next round?

(k) Will the other investors like this venture?

(l) Will they want to invest with you?

(m) Have you identified the investors for the next round?

(n) Did they see the business plan?

(o) Do they like it?

(p) Did they agree to participate in the next round?

(q) Do you expect VC market to be doing well in the next few years?

7. Exit Risk

(a) Do expect to take this company public?

(b) Will it be ready to go public in three years?

(c) Do you expect economy to boom y the time you are ready for an IPO?

(d) Do you expect the market to be on the rise three years from now?

(e) Will the IPO window be open when this venture is ready to go public?

(f) If the IPO is not an option, can you sell this venture?

(g) Do you know any companies that might want to buy this Venture?

(h) Would they buy the Venture for its market share?

(i) Would they buy the Venture for its product?

(j) Would they buy the Venture for its people?

(k) Would they buy the Venture for its intellectual property?

8. Conclusion

(a) Do you like this investment opportunity?

(b) Do you want to invest?

(c) Are you better of investing elsewhere?

(d) Do you understand all the risks involved?

(e) Are you comfortable with the risk factors?

(f) Are you prepared to lose the entire investment?

(g) Are you a gambler?

(h) Did you finish the due diligence?

(i) Do you like what you see?

(j) Are there any other investment opportunities?

(k) Aren't you better of with any of them?

(l) Do you like this deal more?

(m) Is it because of the people?

(n) Is it because of the product?

(o) Have you thought it through?

(p) Are you sure you want to invest?

The physiological responses to the questions are indicative of the problem-solver's unconscious or “intuitive” reactions thereto. While the problem-solver may not consciously know the answers to the questions, general background information, coupled with experience, often combine to provide a subconscious, intuitive “gut reaction” answer. By analyzing physiological responses at step 300, these intuitive answers can be brought to the conscious level. More importantly, where the problem-solver consciously knows the answers to the questions, the physiological response indicates his level of concern, uncertainty or the perceived level of importance of the subject.

When the answers to a sufficient number of the questions have been brought to a conscious level, they can provide the basis for the solution of the subject problem at step 400. The solution of the problem itself may be performed either consciously or subconsciously. The sufficient number may be all of the questions, or some lesser number. If the problem-solver reaches a conscious solution to the problem, no further questioning is required.

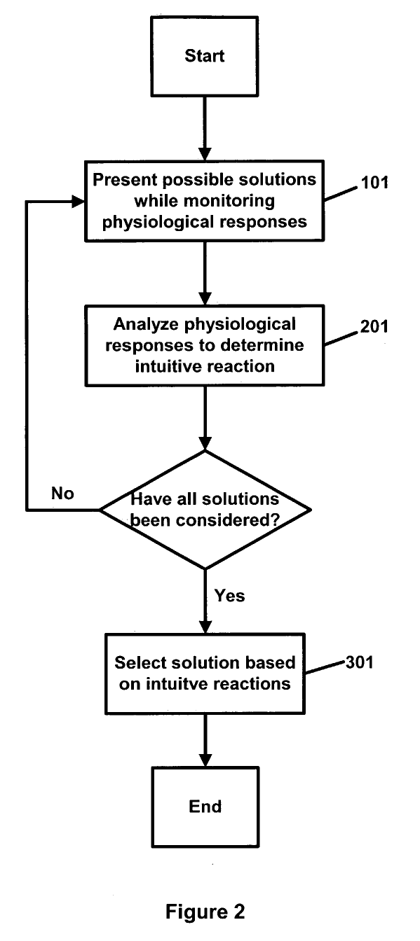

As visually represented in FIG. 2, complex or otherwise intractable problems may also be solved, in accord with the present invention, by serially presenting each of the possible solutions to the problem-solver at step 101, while monitoring the physiological responses thereto.

The physiological responses of the problem-solver to the possible solutions are analyzed at step 301 to determine an intuitive reaction to each. Finally, based upon the intuitive reactions to the various possible solutions to the problem, one is selected at step 201.

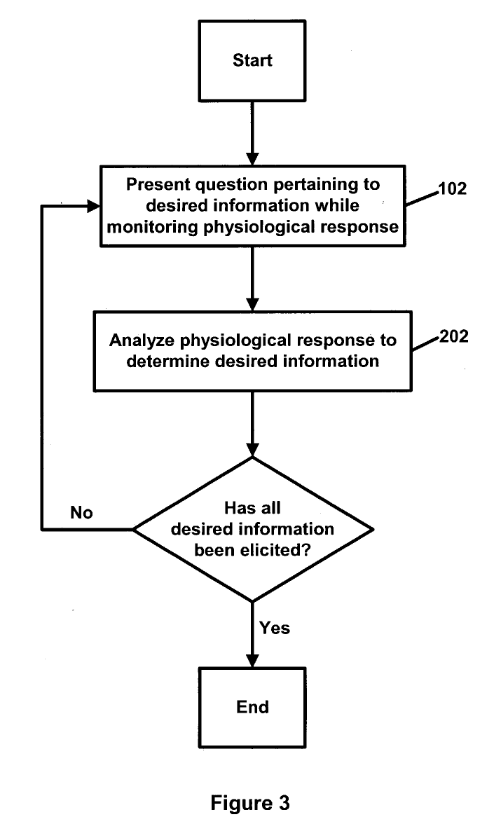

In FIG. 3, there is visually represented a method, in accord with the present invention, for eliciting information held subconsciously by an individual. In step 102, the subject is sequentially asked a series of questions, each pertaining to the desired information, while the physiological responses are being monitored. The responses are analyzed, in step 202, to determine the answers to the questions. The process is continued until all of the desired information has been elicited.

Questions may be presented to the problem-solver manually, i.e. by a human interrogator, either orally or in writing. Alternatively, questions may be presented by a computer. Questions presented by a computer may be prepared in advance by an interrogator, or the problem-solver himself, and loaded into a suitable computer. The questions are then presented to the problem-solver visually, on a monitor screen or a typed print-out, or orally, through the use of voice synthesis circuitry. The problem-solver may respond to the questions either by typing a response on a computer keyboard or by answering audibly to a computer equipped with conventional voice recognition circuit. In computerized applications of the present invention, the sensors, which monitor the physiological responses of the problem-solver, may be connected to the computer, such that the monitoring of the responses are all parts of a fully integrated system.

In lieu of programming a computer to mindlessly present specific questions in a predetermined order, the computer may be programmed to react to the physiological responses to questions. In such an embodiment, the computer may skip certain question or may alter the order of presentation of questions, depending upon the problem-solver's response to previous questions.

In another embodiment, the computer may be programmed with so-called “artificial intelligence”, whereby it prepares and presents questions to the problem-solver without outside intervention.

I claim:

1. A method of solving a problem, comprising the steps of:
a. identifying at least one question pertaining to the problem, an answer to which would bear on the solution to the problem;
b. presenting said at least one question to a problem-solver while monitoring the problem-solver's physiological responses thereto;
c. analyzing said physiological responses to determine the problem-solver's intuitive answer to said at least one question; and
d. solving the problem on the basis of said intuitive answer to said at least one question.

2. The problem-solving method of claim 1, wherein said at least one question comprises a plurality of questions, said questions being presented sequentially, with said physiological response to one of said questions being analyzed before the next said question is presented.

3. A method of harnessing intuition in the solution of a problem, comprising the steps of:
a. monitoring the individual's physiological response thereto;
b. analyzing said physiological responses to determine the individual's intuitive reaction to said possible solutions; and
c. selecting one of said solutions to the problem based upon said intuitive reactions.

4. A method of eliciting, from an individual, desired information held at a subconscious level, the method comprising the steps of:
a. presenting to the individual at least one question pertaining to the information sought to be elicited;
b. monitoring the individual's physiological responses to said at least one question; and
c. determining the desired information on the basis of said physiological responses to said at least one question.

5. The problem-solving method of claim 1, wherein said presenting of said at least one question is performed by a computer.

6. The problem-solving method of claim 1, wherein said monitoring of said physiological responses is performed by a computer.

7. The problem-solving method of claim 1, wherein said analysis of said physiological responses is performed by a computer.

8. The problem-solving method of claim 2, wherein said questions are prepared by a computer utilizing artificial intelligence.
